(19) United States
(12) Patent Application Publication
(10) Pub. No.: US 2012/0290013 A1
(43) Pub. Date: Nov. 15, 2012

(54) TAPERED SPINAL ROD

(76) Inventor: Peter Melott Simonson, Longboat Key, FL (US)

(21) Appl. No.: 13/429,192

(22) Filed: Mar. 23, 2012

Related U.S. Application Data
(60) Provisional application No. 61/467,358, filed on Mar. 24, 2011.

(51) Int. Cl. A61B 17/88; A61B 17/70

(52) U.S. Cl. 606/279; 606/246

(57) ABSTRACT

A tapered spinal rod to support the vertebrae of a spinal column. The cross sectional diameter of one end of the tapered spinal rod is larger than the other end. The cross sectional diameter range of the tapered spinal rod is preferably between 6.5 mm and 3 mm.

### CROSS-REFERENCE TO RELATED APPLICATIONS

This application claims priority to U.S. Provisional Application No. 61/467,358, filed on Mar. 24, 2011, the disclosures of which are incorporated herein by reference in their entirety for all purposes.

### FIELD OF THE INVENTION

The present invention relates to devices, implants and tools used in orthopedic spinal surgical procedures. Specifically, the invention is an improved spinal rod used in the stabilization and fixation of the human spinal column.

### BACKGROUND OF THE INVENTION

Back pain is a commonly reported medical aliment. It is most frequently associated with degenerative changes in the spinal vertebra. Most of the 30 million U.S. patients who report back pain each year resolve their pain with conservative treatment (e.g., rest and exercise). Nonetheless, approximately 15 percent or 4.5 million fail conservative therapy and are left with debilitating pain. Out of these, approximately 500,000 people opt for spinal surgery. In addition to alleviating pain, spinal surgery seeks to minimize damage to adjacent supportive muscle and skeletal components. Although other dynamic treatment options are becoming available, spinal fusion is the most common surgical procedure to address back pain.

Several techniques and systems have been developed for correcting and stabilizing the spine and facilitating spinal fusion. Over the years, spinal and orthopedic implants have evolved toward progressively stronger, stiffer and better devices, as it is presumed that increased construct rigidity optimizes the bone fusion and provides more rapid and robust healing. The most widely used systems use a bendable rod that is placed longitudinally along the length of the spine. Such a rod is bent to follow the normal curvature of the spine whether it is the normal kyphotic curvature for the thoracic region or the lordotic curvature for the lumbar region. In such a procedure, a rod is attached to various vertebrae along the length of the spinal column by a number of bone anchor assemblies. A bone anchor may be a hook that engages the vertebra laminae or a bone screw threaded into the vertebral bone.

In traditional rigid stabilization systems, rods are situated on the opposite sides of the spine or spinous processes. Numerous bone screws are then screwed into the pedicles of the vertebral bodies. Rods are affixed to these bone screws through connectors so corrective and stabilizing forces are applied to the spine. When stabilized, the vertebra is decortified in fusion procedures where the outer cortical bone is removed to provide a foundation for bone grafts. Over time, these bone grafts fuse the damaged vertebrae together.

In the past, commercial spinal rod fixation systems incorporated a number of elongated rods, hooks, screws and bolts that are configured to create segmental constructs through the spine. In earlier versions, spinal rods, vertebral connectors and bone screws were uniform, that is, one-size fit all. In most cases, the same size rod, connectors or bone screws were used throughout the spinal column.

The spine, however, consists of a stack of 33 curved vertebrae that are structurally divided into five regions—cervical region (C1-C7), thoracic region (T1-T12), lumbar region (L1-L5) and, finally, the fused scrum and coccyx regions. Lower down the spine, the vertebrae become larger as the spine supports heavier loads. The cervical vertebrae, forming the neck areas, are relatively small. Just below the cervical vertebrae are the thoracic vertebrae, which form the upper back. The thoracic vertebrae are larger than the cervical vertebrae, and increase in size from top to bottom. Below the thoracic region lies the lumbar vertebrae, which are even larger and support the weight of the entire upper body.

In the past, there were only two spinal rod sizes, a 5.5 mm and 6.36 mm rod. These rod designs and sizes came from the desire to achieve a rod implant with a high level of safety and efficacy in conjunction with a high degree of user friendliness, especially for the surgeon. With such implants, the size, positioning and curvature of the cervical spine, however, presented surgeons with a greater challenge than for the larger lumbar spine. For instance, since the cervical vertebrae are relatively small and spaced close together, the standardized devices used to anchor a spinal rod in this region were relatively large and usually abutted one another. Furthermore, anchoring a 5.5 mm spinal rod to cervical vertebrae with large screws designed for lumbar use may damage the smaller cervical vertebrae. In addition, the gauge or stiffness of the spinal rods used in the cervical region should differ from that used in the thoracic or lumbar regions.

Since these first implants, spinal rod fixation systems have undergone an evolution, that are moving toward lower hardware profile and reduced implant bulk. There are now studies that show that the benefits of small-diameter rods can be obtained without an increased incidence of rod failure. For example, the TSRH® 3Dx™ and the CD Horizon® Legacy™ systems (FIG. 5) sold by Medtronic have a wide variety of hardware that provides anterior or posterior spinal instrumentation in the thoracic and lumbar spine for any pediatric or adult patient requiring spinal stabilization. These instrumentation systems are centered on four different rod diameters, 3.5, 4.5, 5.5 and 6.35 mm. Whereas CD Horizon® Legacy™ includes bone screw sizes ranging from 4.0 to 8.5 mm in 0.5 mm increments, the TSRH® 3Dx™ has bone screws from 4.5 to 8.5 mm in 1 mm increments. Surgeons can now choose a modularity of implant sizes that is best for each patient in each particular operative setting. The 5.5 mm rod, however, is still the most universal system; basically covering any patient ranging from age 10 and up and, thus, is amenable to the majority of adolescent and adult patients requiring spinal instrumentation. In patients undergoing both anterior and posterior instrumentation, a single-rod system such as a dual-rod 5.5 mm system is often used. On the other hand, an anterior-only procedure for a large thoracolumbar/lumbar scoliosis might use only a 4.5 mm dual system.

Presently, there are attempts to meld the rod and bone screw assembly modularity with segmented rods having multiple diameters. FIG. 1, for example, shows an attempt to combine different size rods together with a segmented rod or a transitional rod with cuts or grooves. Although the rod diameter and screw size has been reduced, each segment still needs a similar size bone anchor assembly. In the case of the CD Horizon® Legacy™ in FIG. 1, the tulip bulb bone screw assembly 2 can only take a similar sized rod 4, that is, the larger the rod, the larger the bone screw assembly or vice versa. More recently, there have been further attempts to combine different size rods 6, 8 with connectors 10 between rods (FIG. 2). In both cases, though, there is either additional hardware needed or insufficient bone screw assembly design to achieve perfect modularity between spinal rods and bone screw assemblies. In many cases where an immobilization system has to span the cervical and thoracic and potentially the lumbar vertebrae, the ability to connect a smaller diameter cervical spinal rod with larger diameter thoracic and lumbar spinal rod and connectors presents a problem. In segmented rods with multiple diameters, similar or different sized bone anchor assemblies may not be able to be connected to such rods along the transition portions (FIG. 1).

In summary, there is a need in the industry for an improved spinal rod to lower hardware profile and reduce implant bulk, especially, for the cervical region. The present invention describes such an improvement and achieves a desirable modularity between spinal rods and bone screw assemblies.

### BRIEF SUMMARY OF THE INVENTIONS

The present invention provides an improved spinal rod for orthopedic spinal surgical procedures. Specifically, the preferred embodiment of the present invention is a continuous and gradual tapered spinal rod that allows bone anchor assemblies to be secured almost anywhere along the spinal rod's length.

A preferred embodiment of the present invention is a tapered spinal rod to support the vertebral components of a spinal column. The rod takes a generally continuous cylindrical and gradual tapered shape from one end to the other. The cross section diameter of one end is larger than the other end and is preferably between 6.5 mm and 3 mm. A more preferred embodiment is a tapered spinal rod from 5.5 mm to 3 mm with a cross sectional diameter of 4.5 mm at its mid-point.

In such a preferred embodiment, the tapered spinal rod allows the appropriate cross sectional rod diameter to match the load strength that corresponds to the spinal region to which it is attached, whether it is the cervical region (C1-C7), thoracic region (T1-T12) or lumbar region (L1-L5). In doing so, it lowers and reduces hardware profile and implant bulk.

The tapered spinal rod of the present invention allows bone screw assembly systems to slide continuously on the rod without changing connector size. The same size bone screw assemblies can be used throughout. Alternatively, various smaller or larger bone screw assemblies may be used without interrupting the size of the rod, connector or bone screw.

In another example, the tapered spinal rod of the present invention provides a more easily bendable rod placed along the length of the spine. A tapered spinal rod can be easily bent to follow the normal curvature of the spine whether it is the curvy cervical region, the gradual kyphotic curvature of the thoracic region or the heavy load bearing lordotic curvature of the lumbar region. The present invention avoids bending an overly strong 5.5 cm rod where it is not necessary, especially in the cervical region. The tapered spinal rod can easily span the cervical and thoracic regions, and potentially the lumbar vertebrae as well, providing the ability to connect the smaller diameter cervical section with smaller bone anchor assemblies.

Finally, the present invention completes the modularity evolution of traditional rigid stabilization systems. The single tapered spinal rod of the present invention can now allow appropriate sized bone screw assemblies to be matched with corresponding spinal regions. The present invention also provides a spinal rod implant with a high level of safety and efficacy without the tendency to overload the spine with hardware and implants.

### BRIEF DESCRIPTION OF THE DRAWINGS

FIG. 1 shows a segmented spinal rod with multiple diameters and corresponding connector sizes.

FIG. 2 shows two different sized spinal rods joined by a rod connector.

FIG. 3 and FIGS. 3A-3C show a tapered spinal rod of the present invention with different cross sectional diameters.

FIG. 4 shows perspective view of the TSRH® 3D™ Spinal System described in U.S. Pat. No. 5,643,263 to Simonson.

FIG. 5 compares a CD Horizon bulb type bone anchor assembly to a TSRH® 3Dx™ bone anchor assembly.

FIG. 6 shows a series of same size TSRH® 3Dx™ bone screw assemblies sliding along a tapered spinal rod of the present invention.

FIG. 7 shows a full-length tapered spinal rod with various sized bone screw assemblies that appropriately match the vertebral column region to the proper rod diameter size.

FIG. 8 shows segmented pre-contoured tapered spinal rods with various sized bone screw assemblies.

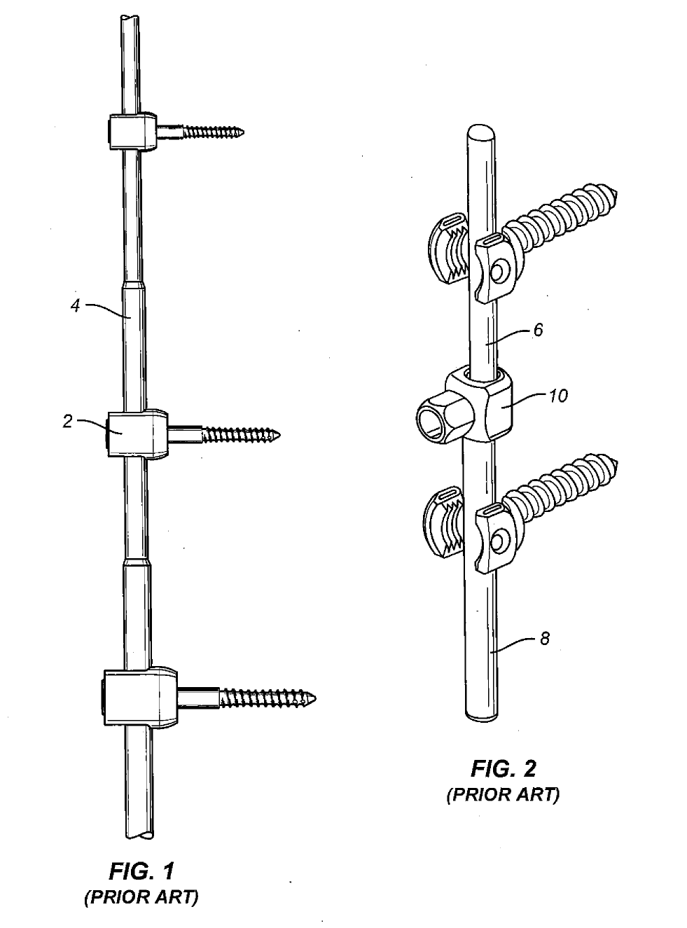

### DETAILED DESCRIPTION OF THE INVENTION

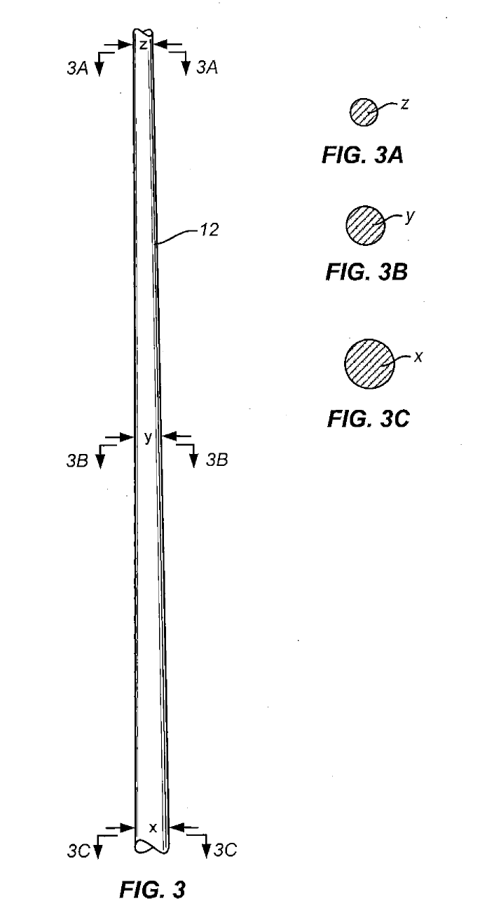

The preferred tapered spinal rod 12 of the present invention shown in FIG. 3 has a continuous and gradual tapered shape. The generally circular cross sectional diameter of one end is larger than that of the other end. The cross sectional diameter range of the tapered spinal rod 12 is preferably between 6.5 mm and 3 mm. A more preferred embodiment is a tapered spinal rod 12 from 5.5 mm to 3 mm with a cross sectional diameter of 4.5 mm at about its mid-point.

As shown in FIGS. 3A, 3B and 3C, different diameters x, y, z are contemplated that continuously differs from one portion of the rod to another portion of the rod. With gradual changes in cross sectional diameters, it is contemplated that three different diameters (FIGS. 3A, 3B & 3C) can be used to match the stiffness, gauge or stress loads necessary to support the three major regions of the spine—cervical region (C1-C7), thoracic region (T1-T12) and lumbar region (L1-L5). These average diameters are preferably 3.2 mm to 4.0 mm for the cervical region and between 4.5 mm to 6.5 mm for both the thoracic and lumbar regions. These preferred ranges may provide a higher level of safety and efficacy in conjunction with a lower hardware profile and reduced implant bulk.

The preferred tapered spinal rod is generally cylindrical in shape with either a round, oval or elliptical cross section geometry. Preferably, the tapered spinal rod is made from metals such as stainless steel, titanium, cobalt chromium alloys, nickel-titanium alloys or other suitable high strength materials. The tapered spinal rod may also be made of polymer materials such as PEEK (polyether ether ketone) or carbon fiber-reinforced polymers where the high strength-to-weight ratio contributes to reduced hardware and implant bulk.

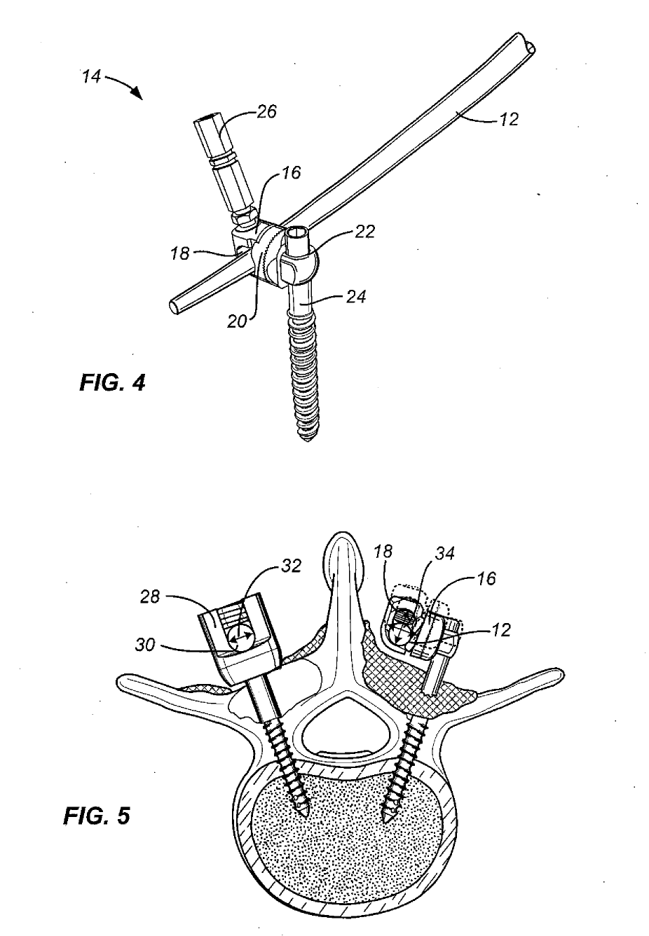

The tapered spinal rod of the present invention works well with bone screw assemblies that can accommodate various rod diameters such as the TSRH® Spinal System described in U.S. Pat. No. 5,643,263 to Simonson. FIG. 4 shows one of the designs of this variable height TSRH® 3D™ Spinal System assembly 14. The assembly 14 consists a rod washer 16 with an oversized aperture 18 and bone washer 20 also with an oversize aperture 22. These oversize apertures 18, 22 or bores can receive a tapered spinal rod 12 or bone screw 24 of any size and thicknesses with different sized interface washers. Using this type of bone screw assembly allows the bore diameters of both the tapered spinal rod 12 and bone screw 24 to be effectively reduced as the tapered spinal rod 12 and washers 16, 20 are pressed together by a top-tightening set screw 26. When the tapered spinal rod 12 and washers 16, 20 have been properly positioned over the rod 12 and bone screw 24, they are tightened by the set screw 26. When the set screw 26 forces the rod 12 toward the bone screw washer 20, the entire assembly becomes locked against any movement. Adjustments can then be made by loosening the set screw 26 and re-tightening it when the preferred position is reached. When properly adjusted, the set screw 26 is tighten and snapped off. To improve the snugness of the fit between the tapered spinal rod 12 and the rod washer 16, the interior surface of the rod washer 16 can be tapered to match the taper of the tapered spinal rod 12. This principle of matching the interior connector surface to the taper of the tapered spinal rod 12 can also apply to other embodiments, such as with bulb connectors 28, cross-linked connectors, co-linear rod extenders or parallel rod extenders.

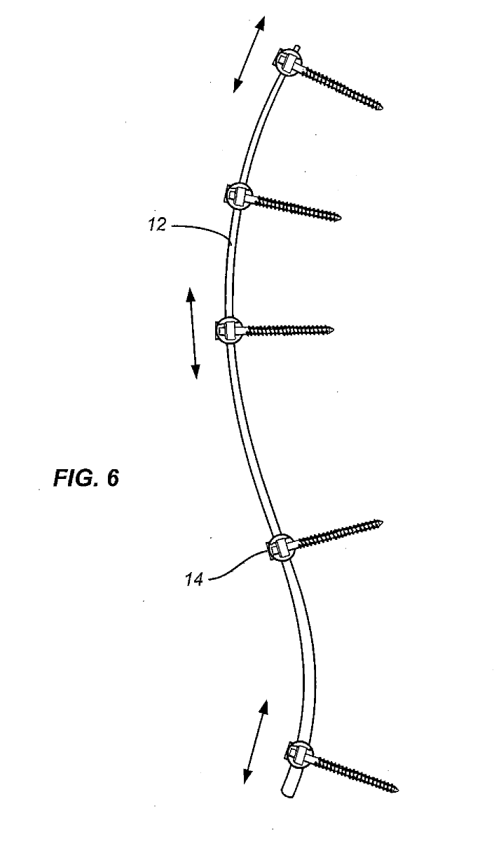

The ability of the TSRH® Spinal System to accept various cross sectional diameters of the tapered spinal rod 12 can be seen more clearly in FIG. 5. While the common CD Horizon bulb type bone anchor assembly 28 has a set width to exactly match the rod 30 diameter 32, the oversize rod washer aperture 18 of the rod washer 16 on the TSRH® 3Dx™ Spinal System with its hexagonal set screw design can accommodate the various cross sectional diameters 34 of the tapered spinal rod 12. In this preferred embodiment, the tapered spinal rod 12 shown in FIG. 6 allows TSRH® 3Dx™ bone screw assemblies 14 to slide easily and continuously up and down on the tapered spinal rod 12 of the present invention without changing the bone screw assembly 14 size.

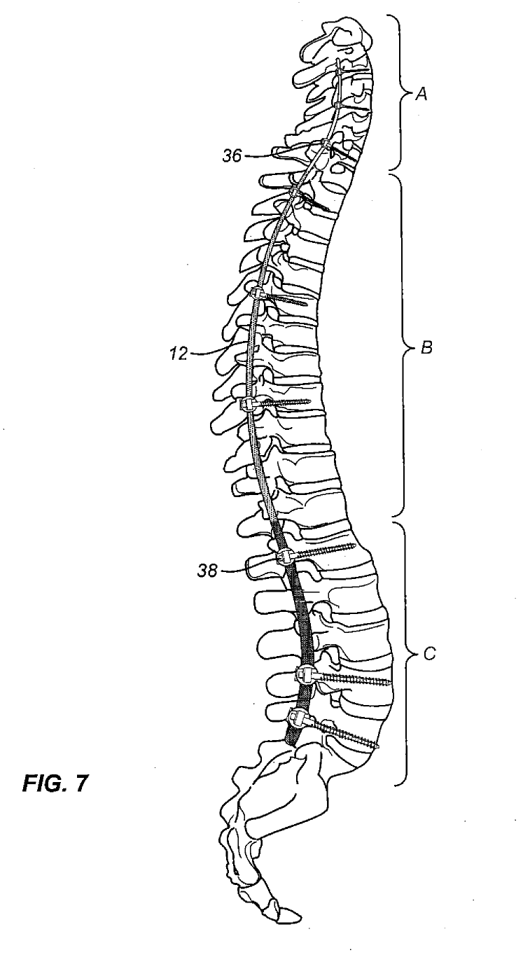

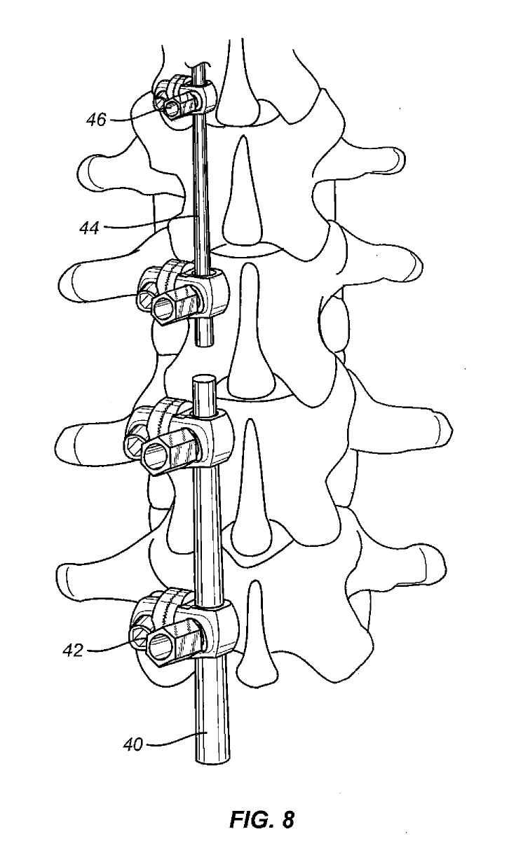

In another embodiment, the tapered spinal rod 12 of the present invention provides a more easily bendable rod placed along the length of the spine. As shown in FIG. 7, a tapered spinal rod 12 can be more easily bent to follow the normal curvature of the spine whether it is the more curvy cervical region A, the gradual kyphotic curvature of the thoracic region B or the heavy load bearing lordotic curvature of the lumbar region C. The tapered spinal rod 12 of the present invention avoids bending an overly strong rod when a 5.5 cm load bearing rod is not necessary, especially in the cervical region A. The tapered spinal rod 12 can also span the cervical and thoracic, and potentially the lumbar vertebrae as well, providing the ability to connect the smaller diameter cervical section with smaller bone anchor assemblies 36. On the other hand, larger TSRH® bone anchor assemblies 38 may be used on the tapered spinal rod 12 in the lumbar region C without interrupting or changing the rod. In this preferred embodiment, the cross sectional diameter or gauge of the tapered spinal rod 12 can be chosen to match the load strength necessary to support the corresponding cervical region A (C1-C7), thoracic region B (T1-T12) and/or lumbar region C (L1-L5). Again, matching the gauge of the tapered spinal rod 12 to the vertebrae region it is intended to support lowers and reduces its hardware profile and implant bulk.

In yet another preferred embodiment also shown in FIG. 7, various sections of tapered spinal rod may be colored differently (shaded areas) to identify various rod segments corresponding to the appropriate cervical, thoracic and lumbar vertebrae regions. Various bone anchor assemblies may also be similarly color-coded to ensure that the appropriate sized bone anchor assembly is correctly matched to its appropriate section on the tapered spinal rod. This embodiment also avoids any confusion of using different or inappropriate spinal rods and bone anchor assemblies with one another.

In another preferred embodiment, the tapered spinal rod may have different material properties to accompany its tapered shape. The tapered spinal rod can start out with a lower strength or less stiff materials at its thicker portion but change metal composition at its thinner portion to provide similar stiffness as its diameter decreases. For example, the tapered spinal rod may be made in different sections from a combination of stainless steel, titanium, cobalt chromium alloys, nickel-titanium alloys or other suitable high strength materials. For example, the tapered spinal rod may be made from a combination of cobalt chromium alloy and titanium metals in different sections to provide a desirable but different combination of strength and stiffness along its length. A lower strength metal such as cobalt chromium alloy may be used at its thicker section and a higher strength and stiffer material such as titanium can be used at its thinner sections. The use of such different metals allows for varying degrees and combinations of strengths and stiffness appropriate to secure and immobilize different vertebrae regions without compromising stiffness or strength. Additionally, a metal tapered spinal rod may be exposed to different heat treatments that will also provide various degrees of strengths and stiffness appropriate to also secure and immobilize different vertebrae regions without compromising strength and stiffness.

In another an exemplary aspect of the present invention, the tapered spinal rod can also be pre-contoured and segmented along the spine using sections or parts of the tapered spinal rod to fulfill the spinal support and load necessary. As shown in FIG. 8, a larger pre-contoured tapered spinal rod 40 with larger bone screw assemblies 42 can be used where large loads or support is needed in combination with a smaller pre-contoured tapered spinal rod 44 with smaller bone screw assemblies 46 in places where less support is needed. Again, the modularity of the tapered pre-contoured spinal rod and bone screw assemblies can be matched accordingly.

In the foregoing specification, the invention has been described with reference to specific preferred embodiments and methods. It will, however, be evident to those of skill in the art that various modifications and changes may be made without departing from the broader spirit and scope of the invention. For example, the shape, composition and diameters of the tapered spinal rod may vary. Accordingly, the specification and drawings are to be regarded in an illustrative, rather than restrictive, sense; the invention being limited only by the appended claims.

What is claimed is:

1. A spinal rod of generally circular cross section which continuously reduces in diameter from one end to the other end.

2. The spinal rod of claim 1 wherein the cross sectional shape of the rod is circular or oval.

3. The spinal rod of claim 1 made from the group consisting of stainless steel, titanium, cobalt chromium alloy and/or nickel-titanium alloys.

4. The spinal rod of claim 1 made from PEEK (polyether ether ketone) or carbon fiber-reinforced polymers.

5. The spinal rod of claim 1 wherein a larger diameter section is affixed to the thoracic and/or lumbar spinal region and a smaller diameter section is affixed to the cervical spinal region.

6. The spinal rod of claim 1 wherein said spinal rod is colored to correspond to an appropriate spinal region.

7. The spinal rod of claim 6 wherein corresponding color-coordinated bone anchor assemblies are used.

8. The spinal rod of claim 1 wherein said spinal rod is pre-contoured.

9. A method for treating a plurality of spine vertebrae regions comprising the steps of
selecting a tapered spinal rod having a generally circular cross section and a larger diameter at one end than the other end;
matching appropriate diameters of said tapered spinal rod to spine vertebrae regions; and
attaching said tapered spinal rod to appropriate-sized bone anchor assemblies attached to said spine vertebrae regions.
